(19) United States
(12) Patent Application Publication
(10) Pub. No.: US 2013/0310275 A1
(43) Pub. Date: Nov. 21, 2013

(54) DIAGNOSIS OF ASYMPTOMATIC LEFT VENTRICULAR SYSTOLIC DYSFUNCTION

(71) Applicants: Fatima Smih, Toulouse (FR); Franck Desmoulin, Toulouse (FR); Michel Galinier, Toulouse (FR); Philippe Rouet, Toulouse (FR)

(72) Inventors: Fatima Smih, Toulouse (FR); Franck Desmoulin, Toulouse (FR); Michel Galinier, Toulouse (FR); Philippe Rouet, Toulouse (FR)

(73) Assignee: Institut National de la Sante et de la Recherche Medicale (INSERM), Paris (FR)

(21) Appl. No.: 13/990,448

(22) PCT Filed: Nov. 30, 2011
(86) PCT No.: PCT/EP2011/071387

§ 371 Date: Aug. 2, 2013

(30) Foreign Application Priority Data
Nov. 30, 2010 (EP) 10290629.4

(51) Int. Cl. C12Q 1/68

(52) U.S. Cl. CPC C12Q 1/6883
USPC 506/9

(57) ABSTRACT

The invention relates a method for diagnosing asymptomatic left ventricular systolic dysfunction in a subject comprising the step a) of: —measuring the level of expression of the genes FECH, TMEM79, FBXW7, NGFB, ALK, UBN1 and SLC43A2 in a biological sample of said subject; or—measuring the level of expression of at least one gene selected from the group consisting of FECH, TMEM79, FBXW7, NGFB, ALK, UBN1 and SLC43A2 in a biological sample of said subject.

### FIELD OF THE INVENTION

The present invention relates to a method for diagnosing asymptomatic left ventricular systolic dysfunction.

### BACKGROUND OF THE INVENTION

The risk for developing heart failure (HF) in Western countries is estimated to be 33%, with a 8 year post-diagnosis mortality rate of 75%. Epidemiologic studies have demonstrated that cardiovascular risk factors such as hypertension, diabetes and obesity are precursors of HF. These factors induce modification of the myocardium structure and lead to functional alterations of the heart including a reduction in the left ventricular ejection fraction (LVEF). Identification of patients at the “pre-heart failure” stage, referred to as asymptomatic left ventricular dysfunction (ALVD), can prevent the development of HF through the initiation of adapted medical and non-medical strategies. Asymptomatic left ventricular systolic dysfunction, common in the general population, leads to a high risk of developing HF. Compared to individuals with normal LVEF, ALVD subjects have a 12-fold increase in the annual rate of hospitalization for first-event HF and a 4-fold increase in the risk of death over a 6-year period.

Currently, asymptomatic left ventricular dysfunction can only be diagnosed by transthoracic echocardiography. ALVD diagnosis thus requires a sophisticated echocardiographic analysis, which is both time-consuming and costly, and is not applicable to the large population of individuals at risk.

There is currently no biomarker available for detecting a subject presenting ALVD. This lack of biomarker(s) is of importance because ALVD is highly prevalent due to the general increase in cardiovascular risk factors. ALVD has become established as a predictive early indicator of severe HF. Follow-up studies have shown that ALVD subjects display an average annual chronic heart failure rate of 4.9 to 20%, with a mortality rate of 5.1 to 10.5%. Such observations were recently confirmed in a 5-year survival rate analysis that showed a death rate of 31% for subjects suffering from ALVD and of 47% for patients with systolic HF.

Therefore, there is a need for a method for identifying ALVD individuals in the general population before they develop HF. There is thus a need for identifying biomarker(s), which are independent of cardiovascular risk factors (such as hypertension, diabetes, obesity, dyslipidemia . . . ) and useful to sort easily ALVD individuals among subjects with cardiovascular risk factors.

### SUMMARY OF THE INVENTION

The inventors have studied the impact of ALVD on the human transcriptome and identified a specific molecular signature based on differential gene expression. They analyzed white blood cell transcriptomes since gene expression patterns in peripheral blood has been validated in humans as a basis for the detection and diagnosis of diseases such as chronic and acute heart failure. Previous works have shown that blood cells share 84% of their transcriptome with the heart and that some gene regulations in blood are similar to other organs such as the heart.

Thus, peripheral blood is likely to become a useful resource in the diagnosis of systemic diseases, selection of treatment methods and disease outcome prediction. The inventors have identified a set of gene(s) that could be used to pre-screen patients for ALVD before time-consuming echocardiographic confirmation of the disease.

The invention thus relates to a method for diagnosing asymptomatic left ventricular systolic dysfunction in a subject comprising the step a) of:
measuring the level of expression of the genes NGFB, TMEM79, FBXW7, FECH, ALK, UBN1 and SLC43A2 in a biological sample of said subject; or
measuring the level of expression of at least one gene selected from the group consisting of NGFB, TMEM79, FBXW7, FECH, ALK, UBN1 and SLC43A2 in a biological sample of said subject.

### DETAILED DESCRIPTION OF THE INVENTION

The inventors have shown that the expression of NGFB, TMEM 79 and FBXW7 were significantly down-regulated in patient presenting an asymptomatic left ventricular systolic dysfunction. The inventors also found that the expression of FECH, ALK, UBN1 and SLC43A2 were significantly increased in the same group.

The invention thus relates to a method for diagnosing asymptomatic left ventricular systolic dysfunction in a subject comprising the step a) of:
measuring the level of expression of the genes, FECH, TMEM79, FBXW7, NGFB, ALK, UBN1 and SLC43A2 in a biological sample of said subject, or
measuring the level of expression of at least one gene selected from the group consisting of FECH, TMEM79, FBXW7, NGFB, ALK, UBN1 and SLC43A2 in a biological sample of said subject.

Preferably, the invention relates to a method for diagnosing asymptomatic left ventricular systolic dysfunction in a subject comprising the step of measuring the level of expression of at least one gene selected from the group consisting of NGFB, TMEM79, FBXW7, FECH, ALK, UBN1 and SLC43A2 in a biological sample of said subject.

As used herein, the term “asymptomatic left ventricular systolic dysfunction” refers to the silent (asymptomatic) preclinical state of a patient with left ventricular ejection fraction <45% defined by echocardiography and with high risk of developing a symptomatic heart failure. Said condition is also referred to as “pre-heart failure”.

“Left Ventricular Ejection Fraction” (“LVEF”) refers to a measure of systolic function of the left ventricle. The ejection fraction is the percentage of blood ejected from the left ventricle with each heart beat.

As used herein, the term “diagnosis” in all its grammatical forms, refers to the process of identifying a medical condition or an asymptomatic condition. In the context of this invention, the term diagnosis refers to the identification of subjects presenting asymptomatic left ventricular systolic dysfunction.

As used herein, the term “subject” refers to an individual with symptoms of and/or suspected of having a left ventricular systolic dysfunction. Preferably, said subject is at risk for having or developing cardiovascular diseases.

As used herein, the term “gene” refers to a nucleic acid fragment that expresses a specific protein, including regulatory sequences preceding (5′ non-coding sequences) and following (3′ non-coding sequences) the coding sequence.

As used herein, the expression “gene of interest according to the invention” or “gene of interest” refers to one of the followings genes: NGFB, TMEM79, FBXW7, FECH, ALK, UBN1 and SLC43A2.

The term “NGFB”, refers to the gene of “nerve growth factor (beta polypeptide)”. The sequence of said gene can be found under the Ensembl accession number ENSG00000134259.

The term “TMEM79” refers to the gene of “transmembrane protein 79”. The sequence of said gene can be found under the Ensembl accession number ENSG00000163472.

The term “FBXW7” refers to the gene of the “F-box and WD repeat domain containing 7”. The sequence of said gene can be found under the Ensembl accession number ENSG00000109670.

The term “FECH” refers to the gene of “ferrochelatase”. The sequence of said gene can be found under the Ensembl accession number ENSG00000066926.

The term “ALK” refers to the gene of “anaplastic lymphoma receptor tyrosine kinase”. The sequence of said gene can be found under the Ensembl accession number ENSG00000171094.

The term “UBN1” refers to the gene of “ubinuclein 1”. The sequence of said gene can be found under the Ensembl accession number ENSG00000118900.

The term “SLC43A2” refers to the gene of “solute carrier family 43, member 2”. The sequence of said gene can be found under the Ensembl accession number ENSG00000167703.

As used herein, the term “gene expression level” or “the expression level of a gene” refers to an amount or a concentration of a transcription product, for instance mRNA, or of a translation product, for instance a protein or polypeptide. Typically, a level of mRNA expression can be expressed in units such as transcripts per cell or nanograms per microgram of tissue. A level of a polypeptide can be expressed as nanograms per microgram of tissue or nanograms per milliliter of a culture medium, for example. Alternatively, relative units can be employed to describe an expression level.

As used herein, the expression “mRNA transcript” refers to the product resulting from RNA polymerase-catalyzed transcription of a DNA sequence without introns and that can be translated into polypeptides by the cell.

As used herein, the term “biological sample” as used herein refers to any biological sample obtained for the purpose of evaluation in vitro. Examples of test samples include blood, serum, plasma, nipple aspirate fluid, urine, saliva, synovial fluid and cephalorachidian liquid (CRL). Preferably, said biological sample is blood, most preferably peripheral blood. In another embodiment, said biological sample is a heart sample, preferably a right auricle appendage.

As used herein, the expression of “measuring the expression level of a gene” encompasses the step of measuring the quantity of a transcription product, preferably mRNA obtained through transcription of said gene, and/or the step of measuring the quantity of translation product, preferably the protein obtained through translation of said gene. Preferably, the step of measuring the expression of a gene refers to the step of measuring the quantity of mRNA obtained through transcription of said gene.

Typically, the step a) of measuring the level of gene expression of said gene(s) may be performed according to the routine techniques, well known of the person skilled in the art.

In one embodiment, step a) of measuring the level of expression of said gene(s) is a step of measuring the expression level of translation products of said gene(s), preferably proteins. Methods for measuring the quantity of protein in a biological sample are well known in the art. Typically, said step may be performed using standard electrophoretic and immunodiagnostic techniques, including immunoassays such as competition, direct reaction, or sandwich type assays. Such assays include, but are not limited to, Western blots; agglutination tests; enzyme-labeled and mediated immunoassays, such as ELISAs; biotin/avidin type assays; radioimmunoassays; immunoelectrophoresis; immunoprecipitation, etc. The reactions generally include revealing labels such as fluorescent, chemiluminescent, radioactive, enzymatic labels or dye molecules, or other methods for detecting the formation of a complex between the antigen and the antibody or antibodies reacted therewith. More preferably, step a) is performed with a fluorescence-activated cell sorter (FACS). Said fluorescence-activated cell sorter is a machine that can rapidly separate the cells in a suspension on the basis of size and the color of their fluorescence.

In a preferred embodiment, said step a) of measuring the level of expression of said gene(s) is a step of measuring the expression level of transcription products of said gene(s), preferably mRNA. Methods for measuring the quantity of mRNA are well known in the art. Typically, the nucleic acid contained in the biological sample may be extracted according to standard methods, for example using lytic enzymes or chemical solutions or extracted by nucleic-acid-binding resins following the manufacturer's instructions. The extracted mRNA may be then detected by hybridization (e.g., Northern blot analysis). Alternatively, the extracted mRNA may be subjected to coupled reverse transcription and amplification, such as reverse transcription and amplification by polymerase chain reaction (RT-PCR), using specific oligonucleotide primers that enable amplification of a region in said genes. Preferably, quantitative or semi-quantitative RT-PCR is used. Extracted mRNA may be reverse-transcribed and amplified, after which amplified sequences may be detected by hybridization with a suitable probe or by direct sequencing, or any other appropriate method known in the art. Other methods of amplification include ligase chain reaction (LCR), transcription-mediated amplification (TMA), strand displacement amplification (SDA) and nucleic acid sequence based amplification (NASBA).

Most preferably, said step a) of measuring the level of expression of said gene(s) are performed by DNA microarray.

As used herein, the expression “microarray” or “DNA microarray” refers to a set of oligonucleotide probes arranged on a solid matrix, such as a microscope slide or silicon wafer. The term “microarray” is thus meant to indicate analysis of many small spots to facilitate large scale nucleic acid analysis enabling the simultaneous analysis of thousands of DNA sequences. This technique is seen as an improvement on existing methods, which are largely based on gel electrophoresis. For a review, see Nature Gen. (1999) 21 Suppl. 1. Line blot assay and microarray methods both use circumscribed areas containing specific DNA fragments. The utility of DNA arrays for genetic analysis has been demonstrated in numerous applications including mutation detection, genotyping, physical mapping and gene-expression monitoring. The basic mechanism is hybridization between arrays of nucleotides and target nucleic acid.

In the context of this invention, the person skilled in the art may use the following probes for carrying out the invention and determining the expression level of the genes of interest according to the invention.

SEQ ID No 1 is a suitable probe for assessing the expression level of NGFB.

SEQ ID No 2 is a suitable probe for assessing expression level of TMEM79.

SEQ ID No 3 is a suitable probe for assessing the expression level of FBXW7.

SEQ ID No 4 is a suitable probe for assessing the expression level of FECH.

SEQ ID No 5 is a suitable probe for assessing the expression level of ALK.

SED ID No 6 is a suitable probe for assessing the expression level of UBN1.

SED ID No 7 is a suitable probe for assessing the expression level of SLC43A2.

As used herein, the expression “target sequence of the gene(s) of the invention” refers to the sequence or a fragment of the sequence of one of the following genes: NGFB, TMEM79, FBXW7, FECH, ALK, UBN1, and SLC43A2.

As used herein, the term “probe” refers to a nucleic acid sequence designed to hybridize specifically to a target sequence of interest. In the context of the present invention, oligonucleotide probes comprise about 50 nucleotides. These probes are thus used to detect the presence of complementary target sequences by hybridization with the target sequences.

In one embodiment, the step a) is a step of measuring the level of expression of at least one gene selected from the group consisting of NGFB, TMEM79, FBXW7, FECH, ALK, UBN1 and SLC43A2 in a biological sample of said subject.

Preferably, the step a) is a step of measuring the level of expression of the gene NGFB.

Preferably, the step a) is a step of measuring the level of expression of the gene TMEM79.

Preferably, the step a) is a step of measuring the level of expression of the gene FBXW7.

Preferably, the step a) is a step of measuring the level of expression of the gene FECH.

Preferably, the step a) is a step of measuring the level of expression of the gene ALK.

Preferably, the step a) is a step of measuring the level of expression of the gene UBN1.

Preferably, the step a) is a step of measuring the level of expression of the gene SLC43A2.

In one embodiment, the step a) is a step of measuring the level of expression of at least two genes selected from the group consisting of NGFB, TMEM79, FBXW7, FECH, ALK, UBN1 and SLC43A2 in a biological sample of said subject.

In another embodiment, the step a) is a step of measuring the level of expression of at least three genes selected from the group consisting of NGFB, TMEM79, FBXW7, FECH, ALK, UBN1 and SLC43A2 in a biological sample of said subject.

In still another embodiment, the step a) is a step of measuring the level of expression of at least four genes selected from the group consisting of NGFB, TMEM79, FBXW7, FECH, ALK, UBN1 and SLC43A2 in a biological sample of said subject.

In a further embodiment, the step a) is a step of measuring the level of expression of at least five genes selected from the group consisting of NGFB, TMEM79, FBXW7, FECH, ALK, UBN1 and SLC43A2 in a biological sample of said subject.

In still a further embodiment, the step a) is a step of measuring the level of expression of at least six genes selected from the group consisting of NGFB, TMEM79, FBXW7, FECH, ALK, UBN1 and SLC43A2 in a biological sample of said subject.

In a most preferred embodiment, the step a) is a step of measuring the level of expression of all the genes of the group consisting of NGFB, TMEM79, FBXW7, FECH, ALK, UBN1 and SLC43A2 in a biological sample of said subject.

In a further embodiment, the step a) is a step of measuring the level of expression in a biological sample of said subject of any of the combinations of genes as follows:

| | | | | | |
|---|---|---|---|---|---|
| NGFB | TMEM79 | | | | |
| NGFB | FBXW7 | | | | |
| NGFB | FECH | | | | |
| NGFB | ALK | | | | |
| NGFB | UBN1 | | | | |
| NGFB | SLC43A2 | | | | |
| TMEM79 | FBXW7 | | | | |
| TMEM79 | FECH | | | | |
| TMEM79 | ALK | | | | |
| TMEM79 | UBN1 | | | | |
| TMEM79 | SLC43A2 | | | | |
| FBXW7 | FECH | | | | |
| FBXW7 | ALK | | | | |
| FBXW7 | UBN1 | | | | |
| FBXW7 | SLC43A2 | | | | |
| FECH | ALK | | | | |
| FECH | UBN1 | | | | |
| FECH | SLC43A2 | | | | |
| ALK | UBN1 | | | | |
| ALK | SLC43A2 | | | | |
| UBN1 | SLC43A2 | | | | |
| NGFB | TMEM79 | FBXW7 | | | |
| NGFB | TMEM79 | FECH | | | |
| NGFB | TMEM79 | ALK | | | |
| NGFB | TMEM79 | UBN1 | | | |
| NGFB | TMEM79 | SLC43A2 | | | |
| NGFB | FBXW7 | FECH | | | |
| NGFB | FBXW7 | ALK | | | |
| NGFB | FBXW7 | UBN1 | | | |
| NGFB | FBXW7 | SLC43A2 | | | |
| NGFB | FECH | ALK | | | |
| NGFB | FECH | UBN1 | | | |
| NGFB | FECH | SLC43A2 | | | |
| NGFB | ALK | UBN1 | | | |
| NGFB | ALK | SLC43A2 | | | |
| NGFB | UBN1 | SLC43A2 | | | |
| TMEM79 | FBXW7 | FECH | | | |
| TMEM79 | FBXW7 | ALK | | | |
| TMEM79 | FBXW7 | UBN1 | | | |
| TMEM79 | FBXW7 | SLC43A2 | | | |
| TMEM79 | FECH | ALK | | | |
| TMEM79 | FECH | UBN1 | | | |
| TMEM79 | FECH | SLC43A2 | | | |
| TMEM79 | ALK | UBN1 | | | |
| TMEM79 | ALK | SLC43A2 | | | |
| TMEM79 | UBN1 | SLC43A2 | | | |
| FBXW7 | FECH | ALK | | | |
| FBXW7 | FECH | UBN1 | | | |
| FBXW7 | FECH | SLC43A2 | | | |
| FBXW7 | ALK | UBN1 | | | |
| FBXW7 | ALK | SLC43A2 | | | |
| FBXW7 | UBN1 | SLC43A2 | | | |
| FECH | ALK | UBN1 | | | |
| FECH | ALK | SLC43A2 | | | |
| ALK | UBN1 | SLC43A2 | | | |
| NGFB | TMEM79 | FBXW7 | FECH | | |
| NGFB | TMEM79 | FBXW7 | ALK | | |
| NGFB | TMEM79 | FBXW7 | UBN1 | | |
| NGFB | TMEM79 | FBXW7 | SLC43A2 | | |
| NGFB | FBXW7 | FECH | ALK | | |
| NGFB | FBXW7 | FECH | UBN1 | | |
| NGFB | FBXW7 | FECH | SLC43A2 | | |
| NGFB | FECH | ALK | TMEM79 | | |
| NGFB | FECH | ALK | UBN1 | | |
| NGFB | FECH | ALK | SLC43A2 | | |
| NGFB | ALK | UBN1 | TMEM79 | | |
| NGFB | ALK | UBN1 | FBXW7 | | |
| NGFB | ALK | UBN1 | SLC43A2 | | |
| NGFB | UBN1 | SLC43A2 | TMEM79 | | |
| NGFB | UBN1 | SLC43A2 | FBXW7 | | |
| NGFB | UBN1 | SLC43A2 | FECH | | |
| NGFB | TMEM79 | FECH | UBN1 | | |
| NGFB | TMEM79 | FECH | SLC43A2 | | |
| NGFB | TMEM79 | ALK | UBN1 | | |
| NGFB | TMEM79 | ALK | SLC43A | | |
| NGFB | TMEM79 | UBN1 | FECH | | |
| NGFB | TMEM79 | UBN1 | SLC43A2 | | |
| NGFB | TMEM79 | SLC43A2 | FECH | | |
| NGFB | TMEM79 | SLC43A2 | ALK | | |
| NGFB | FBXW7 | ALK | SLC43A2 | | |
| NGFB | FECH | UBN1 | ALK | | |
| TMEM79 | FBXW7 | FECH | ALK | | |
| TMEM79 | FBXW7 | FECH | UBN1 | | |
| TMEM79 | FECH | ALK | UBN1 | | |
| TMEM79 | FECH | ALK | SLC43A2 | | |
| TMEM79 | FECH | UBN1 | SLC43A2 | | |
| TMEM79 | ALK | UBN1 | SLC43A2 | | |
| TMEM79 | UBN1 | SLC43A2 | FBXW7 | | |
| TMEM79 | UBN1 | SLC43A2 | FECH | | |
| TMEM79 | ALK | SLC43A2 | FBXW7 | | |
| TMEM79 | ALK | SLC43A2 | FECH | | |
| TMEM79 | ALK | SLC43A2 | UBN1 | | |
| TMEM79 | FBXW7 | ALK | FECH | | |
| TMEM79 | FBXW7 | ALK | UBN1 | | |
| TMEM79 | FBXW7 | ALK | SLC43A2 | | |
| TMEM79 | FBXW7 | UBN1 | FECH | | |
| TMEM79 | FBXW7 | UBN1 | ALK | | |
| TMEM79 | FBXW7 | UBN1 | SLC43A2 | | |
| TMEM79 | FBXW7 | SLC43A2 | FECH | | |
| TMEM79 | FBXW7 | SLC43A2 | ALK | | |
| TMEM79 | FBXW7 | SLC43A2 | UBN1 | | |
| TMEM79 | FECH | SLC43A2 | FBXW79 | | |
| FBXW7 | FECH | ALK | UBN1 | | |
| FBXW7 | FECH | ALK | SLC43A2 | | |
| FBXW7 | ALK | UBN1 | SLC43A2 | | |
| FBXW7 | UBN1 | SLC43A2 | FECH | | |
| FBXW7 | FECH | UBN1 | SLC43A2 | | |
| FBXW7 | FECH | ALK | UBN1 | | |
| ALK | UBN1 | SLC43A2 | FBXW7 | | |
| ALK | UBN1 | SLC43A2 | FECH | | |
| NGFB | TMEM79 | FBXW7 | FECH | ALK | |
| NGFB | TMEM79 | FBXW7 | FECH | UBN1 | |
| NGFB | TMEM79 | FBXW7 | FECH | SLC43A2 | |
| NGFB | TMEM79 | FBXW7 | ALK | UBN1 | |
| NGFB | TMEM79 | FBXW7 | UBN1 | SLC43A2 | |
| NGFB | TMEM79 | FBXW7 | ALK | SLC43A2 | |
| NGFB | FBXW7 | FECH | ALK | UBN1 | |
| NGFB | FBXW7 | FECH | ALK | SLC43A2 | |
| NGFB | FBXW7 | FECH | UBN1 | SLC43A2 | |
| NGFB | ALK | UBN1 | SLC43A2 | TMEM79 | |
| NGFB | ALK | UBN1 | SLC43A2 | FBXW7 | |
| TMEM79 | FBXW7 | FECH | ALK | UBN1 | |
| TMEM79 | FBXW7 | FECH | ALK | SLC43A2 | |
| TMEM79 | FECH | ALK | UBN1 | SLC43A2 | |
| TMEM79 | ALK | UBN1 | SLC43A2 | FBXW7 | |
| TMEM79 | FBXW7 | FECH | UBN1 | SLC43A2 | |
| FBWX79 | FECH | ALK | UBN1 | TMEM79 | |
| FBWX79 | FECH | ALK | UBN1 | SLC43A2 | |
| NGFB | TMEM79 | FBXW7 | FECH | ALK | UBN1 |
| NGFB | TMEM79 | FBXW7 | FECH | ALK | SLC43A2 |
| NGFB | FBXW7 | FECH | ALK | UBN1 | SLC43A2 |
| TMEM79 | FBXW7 | FECH | ALK | UBN1 | SLC43A2 |
| TMEM79 | FECH | ALK | UBN1 | SLC43A2 | NGFB |
| TMEM79 | FBXW7 | ALK | UBN1 | SLC43A2 | NGFB |
| TMEM79 | FBXW7 | FECH | UBN1 | SLC43A2 | NGFB |

The method of the invention may comprise a step b), further to step a) of determining the expression profile of said gene(s). Indeed, once expression levels are determined, an expression profile can be created. Typically, expression profile is obtained with the expression level(s) of one or several gene(s), preferably several genes. The expression profiles are highly convenient for simultaneously comparing the expression level of several genes.

As used herein, the term “expression profile” refers to quantitative and qualitative expression of one or more genes in a sample. The expression profile of a single gene corresponds to the expression level of said gene.

The expression profile is a repository of the expression level data that can be used to compare the expression levels of different genes, in whatever units are chosen. The term “profile” is also intended to encompass manipulations of the expression level data derived from a cell, tissue or individual. For example, once relative expression levels are determined for a given set of genes, the relative expression levels for that cell, tissue or individual can be compared to a standard to determine if expression levels are higher or lower relative to the same genes in a standard. Standards can include any data deemed by one of skilled in the art to be relevant for comparison, for example determined threshold value or expression profile of a positive and/or negative control.

In a preferred embodiment, the method of the invention further comprises a step c), further to step b) of comparing the expression profile obtained in step b) with threshold value(s).

Alternatively, the method of the invention further comprises a step c′), further to step b) of comparing the expression profile obtained in step b) with the expression profile of said gene(s) of interest obtained for at least one control selected from the group consisting of a positive control and a negative control.

This step of comparing the expression profile obtained in step b) to a threshold value or to the expression profiles of a control is useful to identify subjects presenting asymptomatic left ventricular systolic dysfunction.

As used herein, the expression “comparing the expression profile” in all its grammatical forms, refers to the evaluation of the quantitative and/or qualitative difference in expression of a gene. Typically, the person skilled in the art may compare the level of expression of a gene to a control value or threshold value.

Typically, a “control value” or “threshold value” or “cut-off value” can be determined experimentally, empirically, or theoretically. A threshold value can also be arbitrarily selected based upon the existing experimental and/or clinical conditions, as would be recognized by person skilled in the art. Preferably, the person skilled in the art may compare the expression profile of the gene(s) of interest according to the invention with threshold value(s) for said gene(s).

For each gene to be compared to a threshold value, the skilled person in the art will compare the level of expression of said gene to a threshold value.

The inventors have shown that expressions of NGFB, TMEM79 and FBXW7 are down regulated in a subject presenting an asymptomatic left ventricular dysfunction. The inventors have also shown that the expressions of FECH, ALK, UBN1 and SLC43A2 are increased in subject presenting an asymptomatic left ventricular dysfunction.

In another embodiment, the step c′) is a step of comparing the expression profile obtained in step b) with the expression profile of at least one control chosen in the group consisting of a positive control and a negative control.

In this particular embodiment, said positive control is preferably a subject suffering from chronic heart failure, such as chronic stable systolic heart failure or a subject which is known to have symptomatic left ventricular systolic dysfunction. Most preferably, said positive control is the expression profile of a subject which is known to have symptomatic left ventricular systolic dysfunction.

Preferably, said negative control is a healthy subject. Said healthy subject does not suffer from any heart failure or echocardiographic abnormality.

The expression profile of the gene(s) of interest of the present invention is set for said positive and negative controls. The person skilled in the art is thus able to compare the expression profile of the gene(s) of interest in the biological sample of said subject to the expression profile of a positive and/or a negative control. Such comparison will then lead the person skilled in the art to determine if said subject presents asymptomatic left ventricular systolic dysfunction.

In one embodiment, the method of the invention further comprises a step of transthoracic echocardiography of said subject. Said step may confirm the diagnosis obtained by the method of the invention.

### A Specific Embodiment of the Invention

In a particular embodiment of the invention, the invention relates to method for diagnosing asymptomatic left ventricular systolic dysfunction in a subject comprising the step of measuring the level of expression of the genes NGFB, TMEM79, FBXW7, FECH, ALK, UBN1 and SLC43A2 in a biological sample of said subject, said step of measuring the level of expression being a DNA microarray performed with the probes of SEQ ID No 1 to 7.

In this particular embodiment of the invention, the person skilled in the art may use relative level of expression of a gene. Indeed, extracting biological information from microarray data requires appropriate statistical methods. Normalization of gene expression data refers to the comparison of expression levels using reference standards that are consistent across all conditions of an experiment.

As used herein “relative level of expression of a gene” refers to the value of a ratio between the level of expression of a gene and a reference value of level of expression of said gene. Typically, said relative level of expression of a gene is expressed without any unit. Preferably, said relative level of expression of a gene is transformed in a log(base2) value.

As used herein, “reference value of the level of expression of a gene” refers to a level of expression of a gene obtained by statistical analysis well known by the person skilled in the art. In the context of the present invention, the inventors have identified a reference value of level of expression for each of the genes according to the invention. Indeed, the inventors used 4 different populations as follows:
healthy volunteers (HI);
individuals with cardiovascular risk factors with LVEF≧45%;
subjects presenting asymptomatic left ventricular dysfunction (ALVD); and
subjects with stable systolic chronic heart failure (CHF) with LVEF<45%.

The inventors measured the level of expression of each of the genes of interest according to the invention for each of the individuals composing the four above mentioned groups. Starting from the latter and using standard statistical analysis, the inventors defined a reference value of level of expression for each of the genes of interest. Such reference value is highly appropriate for determining the relative level of expression of a gene in subject suspected to present an asymptomatic left ventricular dysfunction.

The relative level of expression of a gene is highly appropriate since it can show any variation in the level of expression of said gene in a subject compared to a reference value.

To facilitate an accurate comparison of the level of expression of a gene in a subject, the person skilled in the art may use a log(base2) value of said relative level of expression of a gene. Log values are highly helpful for transforming the relative gene expression into a linear function. The statistical analysis is therefore easier.

Preferably, in the context of the invention, the person skilled in the art will compare the relative level of expression of a gene to a threshold value, said relative level and said threshold value being both expressed in log(base2).

The inventors have indeed established threshold values for the 7 genes of interest according to the invention. For each gene to be compared to a threshold value, the skilled person in the art will compare the relative level of expression of said gene to said threshold value, both being expressed in log(base 2). Therefore, the step of comparison of the log(base 2) of the relative level of expression of a gene to a threshold is dependent on the nature of the technique used for determining said level of expression. In the context of the invention, said threshold values are highly adapted and appropriate for being compared to relative level of expression, obtained by DNA microarray.

The inventors have shown that expressions of NGFB, TMEM79 and FBXW7 are down regulated in a subject presenting an asymptomatic left ventricular dysfunction.

Preferably, the relative level of expression of the gene NGFB is to be compared to a threshold value comprised between about 0.30 and about 0.40, preferably between about 0.32 and about 0.36, preferably between about 0.33 and about 0.35, and most preferably said threshold value is about 0.34.

Preferably, the relative level of expression of the gene TMEM79 is to be compared to a threshold value comprised between about 0.10 and about 0.20, preferably between about 0.10 and about 0.14, preferably between about 0.11 and about 0.13, and most preferably said threshold value is about 0.12.

Preferably, the relative level of expression of the gene FBXW7 is to be compared to a threshold value comprised between 0.01 and about 0.10, preferably between about 0.01 and about 0.05, preferably between about 0.02 and about 0.04, and most preferably said threshold value is about 0.04.

The inventors have shown that the expression of FECH, ALK, UBN1 and SLC43A2 are increased in subject presenting an asymptomatic left ventricular dysfunction.

Preferably, the relative level of expression of the gene FECH is to be compared to a threshold value comprised between 0.01 and about 0.10, preferably between about 0.02 and about 0.06, preferably between about 0.03 and about 0.05, and most preferably said threshold value is about 0.04.

Preferably, the relative level of expression of the gene ALK is to be compared to a threshold value comprised between about 0.83 and about 1.13 preferably between about 0.86 and about 1.09, preferably between about 0.99 and about 1.03, and most preferably said threshold value is about 1.02.

Preferably, the relative level of expression of the gene UBN1 is to be compared to a threshold value comprised between 0.01 and about 0.50, preferably between about 0.10 and about 0.40, preferably between about 0.15 and about 0.25, and most preferably said threshold value is about 0.23.

Preferably, the relative level of expression of the gene SLC43A2 is to be compared to a threshold value comprised between 0.01 and about 0.10, preferably between about 0.02 and about 0.07, and most preferably said threshold value is about 0.03.

Those thresholds values were identified by the inventors as providing the best results regarding the specificity and sensibility of the method of the invention. Hence, those thresholds give excellent results for the method to avoid false negative and false positive.

The comparison to the relative level of expression of the gene of interest according to the invention thus allow the physician to sort out subject presenting asymptomatic left ventricular dysfunction

### FIGURES LEGENDS

FIG. 1: Flow chart of recruitment protocol involving 294 subjects and overall study design

Healthy volunteers (HI) were recruited from the general population, individuals with cardiovascular risk factors (RF) were from the atherosclerosis prevention center and patients with chronic heart failure (CHF) were recruited from the cardiology department at Rangueil Hospital, Toulouse. All subjects underwent transthoracic echocardiography for left ventricular ejection fraction (LVEF) assessment. We used a threshold value of LVEF<45% to sort individuals into 4 groups: HI, RF with LVEF≧45%, subject presenting asymptomatic left ventricular dysfunction (ALVD), and CHF with LVEF<45%. The inventors identified 9 ALVD cases out of the 128 subjects tested with cardiovascular risk factors. They used the set of cardiovascular risk factors based on the characteristics of the ALVD subjects to match the 4 study groups (n=9). White blood cell gene expression profiling was performed using pangenomic microarrays for all 4 groups. Data were statically analyzed using unsupervised (PCA). Then to build an ALVD classifier model, the nearest centroid classification method (NCCM) was used with the ClaNC software package. Classification performance was determined using the leave-one-out cross-validation method. Expression levels of 7 genes capable of discriminating ALVD were compared between the 4 groups and each gene's capability to discriminate patients with LVEF<45% was evaluated using Receiver Operating Characteristic (ROC) analysis.

FIG. 2: Expression levels for 7 genes discriminate the ALVD group

Relative expression levels of the 7 genes, sorted by the nearest centroid classifier, are assessed for HI (Blank square), RF (grey square), ALVD (dashed and grey square) and CHF (blank dashed square) groups. The box plot presents the median, lower and upper quantiles (25^{th}, 75^{th} percentiles) lower and upper whiskers represent the 10th and 90th percentiles. * P<0.05 where indicated, estimated by one-way ANOVA.

For each gene, the first square from the left correspond to HI group, the second to RF group, the third to ALVD group and the fourth to CHF group.

FIG. 3: Receiver-operating characteristic (ROC) analysis

Receiver-operating characteristic (ROC) analysis of ALVD discriminant genes including HI, HFRF (LVEF≧45%) vs ALVD and CHF (LVEF<45%) groups. The area under the curve (AUC), ranged from 0.78 to 0.92 for predicting ALVD.
a—corresponds to the ROC analysis for the ALK gene;
b—corresponds to the ROC analysis for the UBN1 gene;
c—corresponds to the ROC analysis for FBXW7 gene;
d—corresponds to the ROC analysis for TMEM79 gene;
e—corresponds to the ROC analysis for NGFB gene;
f—corresponds to the ROC analysis for FECH gene; and
g—corresponds to the ROC analysis for SLC43A2 gene.

### EXAMPLE

### Materials and Method

### Patient Inclusions

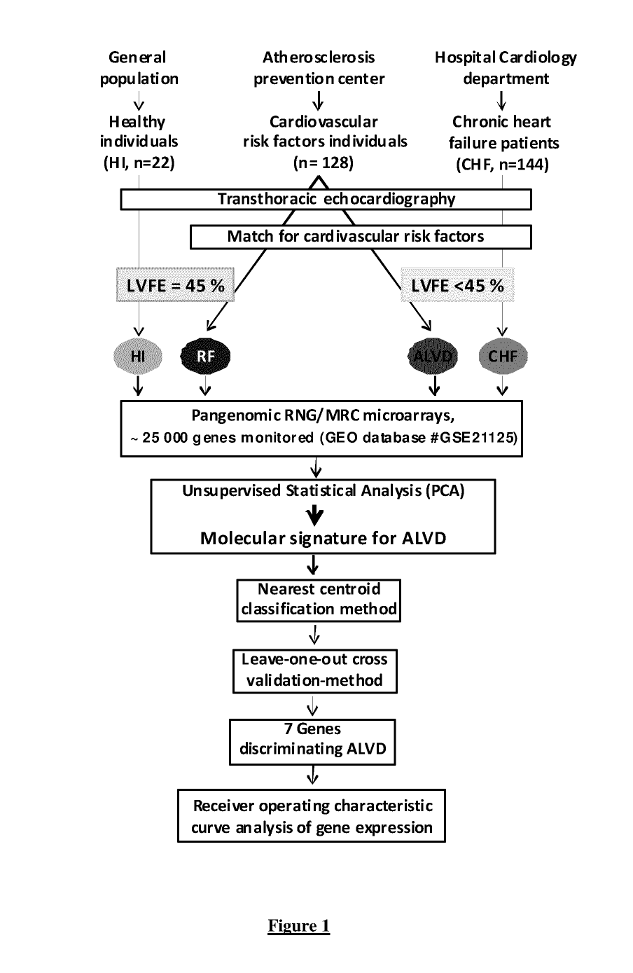

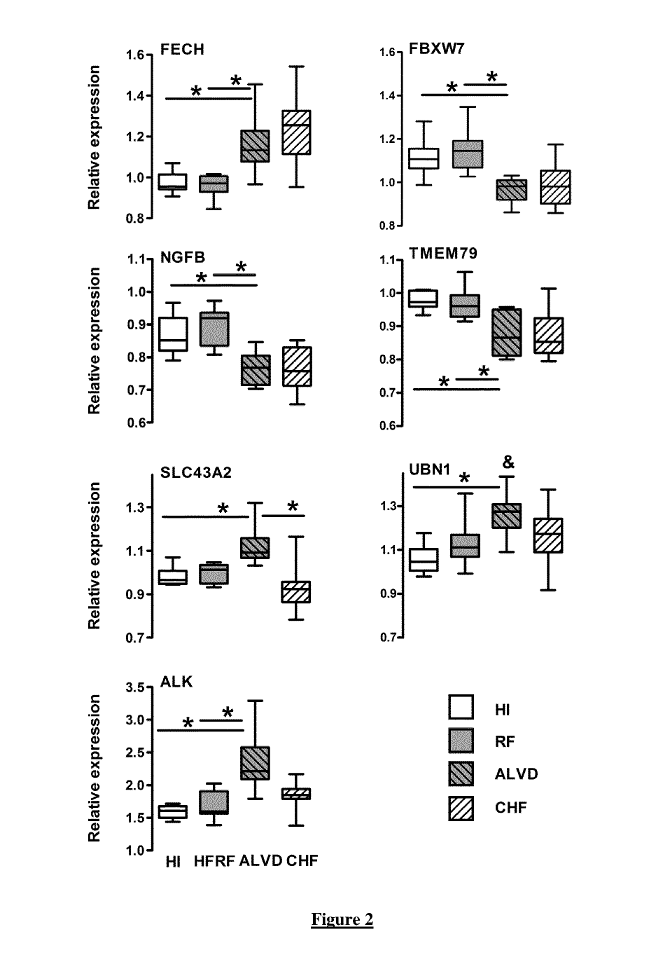

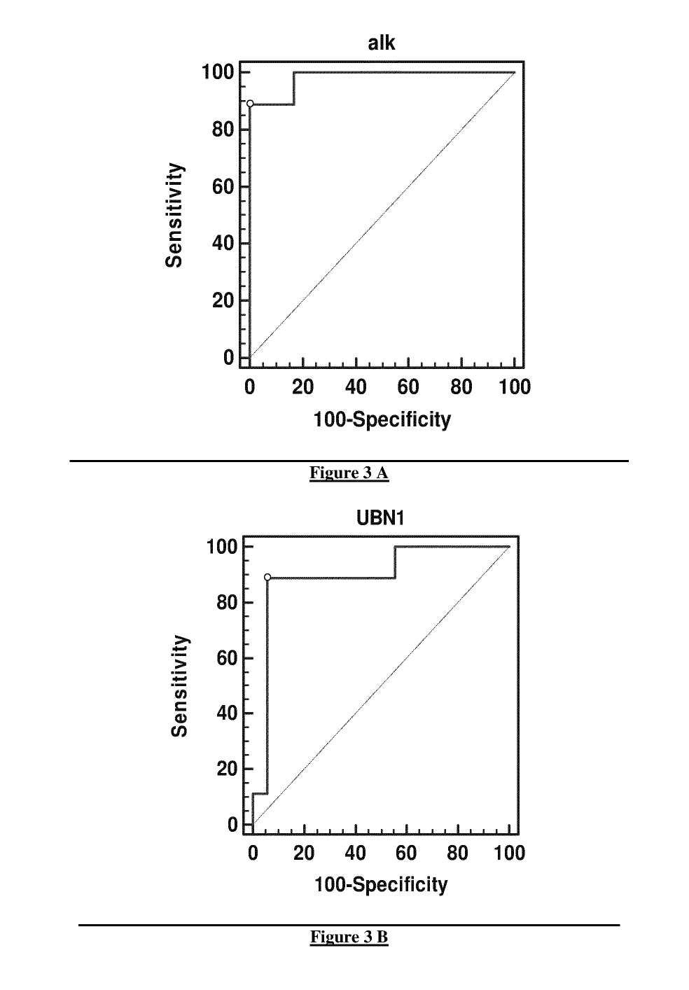

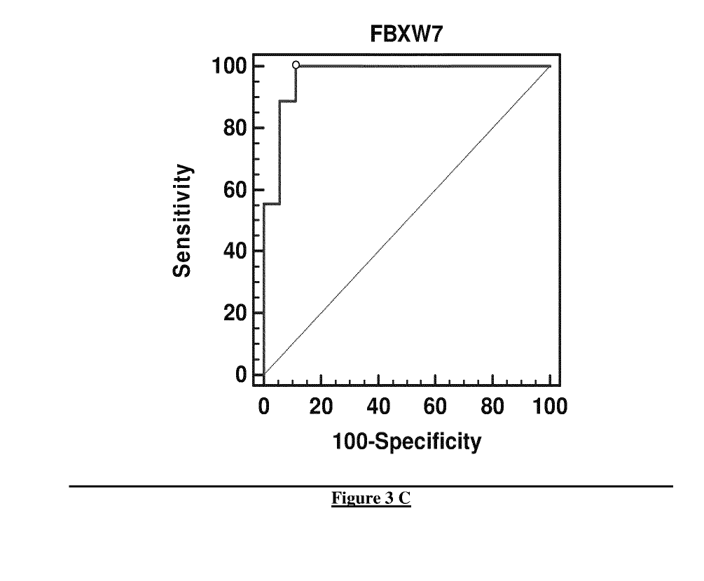

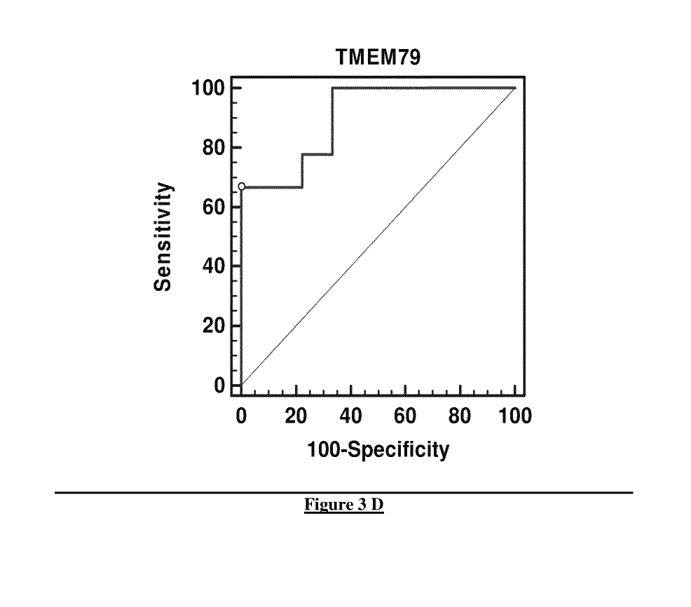

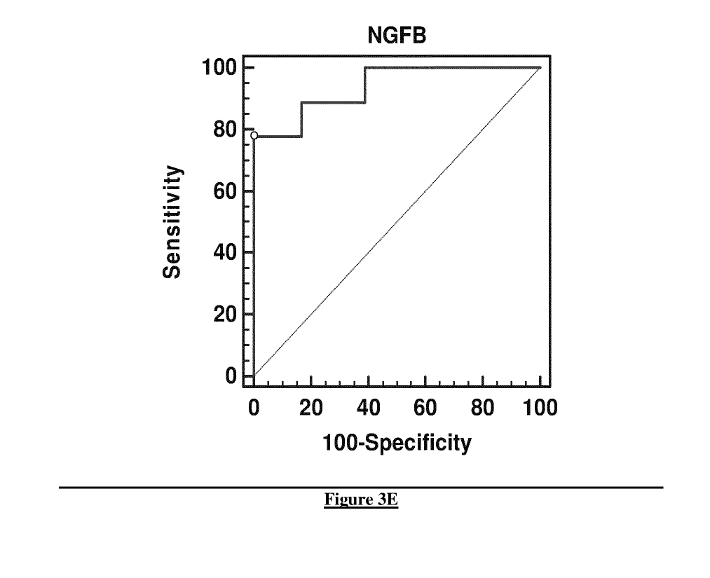

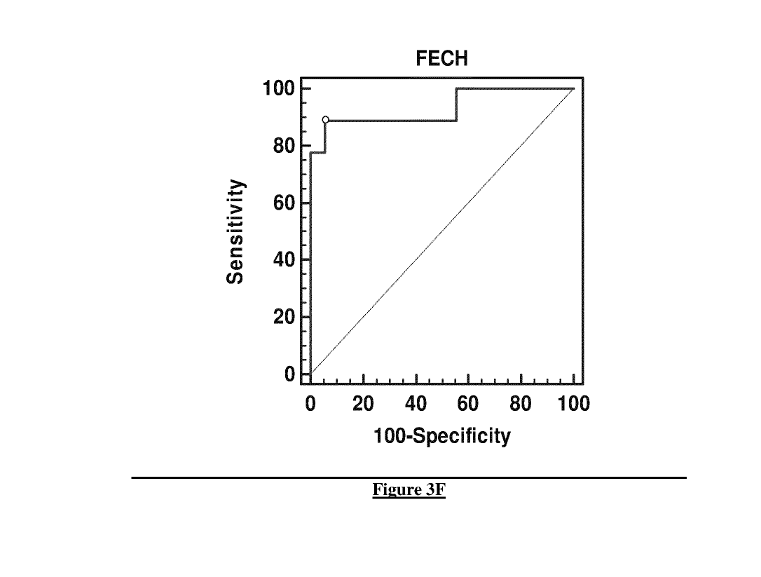

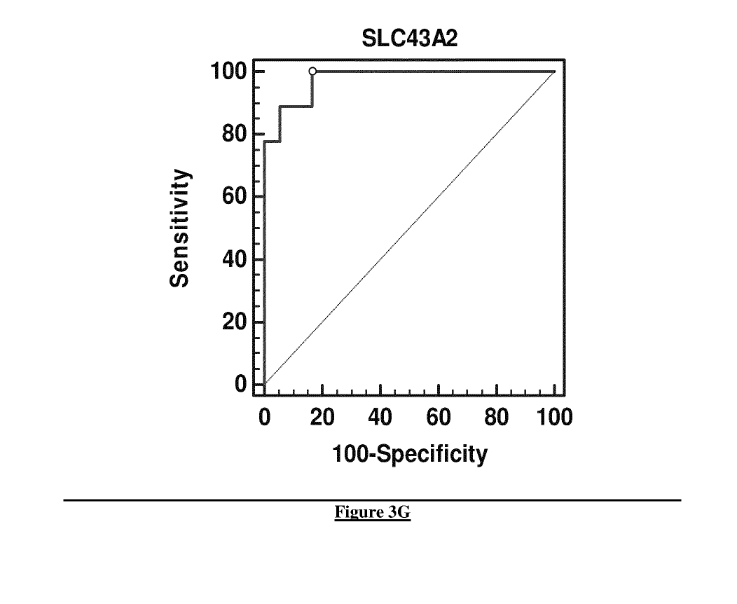

All the 294 subjects underwent transthoracic echocardiography for left ventricular ejection fraction (LVEF) assessment (FIG. 1). Healthy volunteers (HI) without cardiovascular risk or echocardiographic abnormalities were recruited from the general population. Individuals with cardiovascular risk factors and normal left ventricular ejection fraction (RF) and individuals with cardiovascular risk factors and asymptomatic abnormal left ventricular ejection fraction (ALVD) were from the atherosclerosis prevention center. Patients with stable systolic chronic heart failure (CHF) were recruited from the cardiology department at Rangueil Hospital, Toulouse. The inventors used a threshold value of LVEF<45% to sort individuals into 4 groups:
HI,
RF with LVEF≧45%,
ALVD, and
CHF with LVEF<45%.

They identified 9 ALVD cases out of the 128 subjects tested with cardiovascular risk factors. The set of cardiovascular risk factors used to match the study groups were determined based on the characteristics of the ALVD subjects (n=9): mean age 58 years old, 78% male, 44% hypertensive, 33% diabetes, 44% obesity, 56% dyslipidemia and 22% heredity.

The inventors defined two comparative groups:
1—(RF) individuals with cardiovascular risk factors and normal left ventricular ejection fraction (62<LVEF<81%);
2—(ALVD) individuals with cardiovascular risk factors and systolic ALVD (32<LVEF<44%).

Two additional groups were used as controls:
3—(HI, negative control) Healthy volunteers without cardiovascular risk or echocardiographic abnormalities; and
4—(CHF, positive control) patients with chronic stable systolic heart failure (18<LVEF<44%). Moreover, all individuals were extensively phenotyped to check for clinical and biochemical parameters as shown in table 1.

TABLE 1
Cardiovascular risk factors, clinical and biochemical parameters of study groups

| Groups | HI | RF | ALVD | CHF |
|---|---|---|---|---|
| LVEF % | 73 (65-81) | 71 (62-81)* | 39 (32-44) | 33 (18-44) |
| Age (years) | 55 (72-45) | 55 (69-38) | 58 (84-31) | 55 (83-23) |
| Male % | 78 | 78 | 78 | 67 |
| Hypertensive % | 0 | 44 | 44 | 44 |
| Diabetes % | 0 | 33 | 33 | 33 |
| Obesity % | 0 | 44 | 44 | 56 |
| Dyslipidemia % | 0 | 56 | 56 | 44 |
| Heredity % | 0 | 33 | 22 | 11 |
| BMI | 24 ± 3 | 28 ± 3 | 29 ± 4 | 30 ± 5 |
| Systolic blood | 128 ± 16 | 138 ± 12 | 123 ± 17 | 124 ± 19 |
| pressure (mm Hg) | | | | |
| Diastolic blood | 81 ± 9 | 80 ± 14 | 75 ± 12 | 76 ± 9 |
| pressure (mm Hg) | | | | |
| Etiologies | | | | |
| Ischemic % | — | — | — | 33 |
| Hypertensive % | — | — | — | 11 |
| Valvular % | — | — | — | 11 |
| Idiopatic dilated % | — | — | — | 44 |
| Alcool abuse % | — | — | — | 0 |
| Atrial fibrillation % | — | — | — | 33 |
| Labs (mean ± s.d.) | | | | |
| BNP (pg/ml) | 11 ± 9 | 15 ± 10 | 27 ± 23 | 164 ± 151⁼ |
| Na+ (mM) | 140 ± 1 | 140 ± 1 | 139 ± 2 | 138 ± 4 |
| creatinine (μM) | 80 ± 6 | 83 ± 9 | 96 ± 23 | 126 ± 73 |
| Hb (g/dl) | 14.7 ± 1.1 | 14.3 ± 1.1 | 14.1 ± 1.8 | 13.4 ± 1.8 |
| Leukocytes (cells/μl) | 5735 ± 1225 | 6159 ± 1433 | 6765 ± 1533 | 8571 ± 2734 |
| Lymphocytes (cells/μl) | 1865 ± 412 | 1988 ± 428 | 1918 ± 499 | 1898 ± 677 |
| Neutrophils (cells/μl) | 3498 ± 898 | 3630 ± 1073 | 4107 ± 1153 | 6049 ± 2619 |

In this table 1, proportion of individuals with the indicated risk factor for each of the 4 groups of the study is indicated as percentages or averaged value:
HI, Healthy volunteers without cardiovascular risk or echocardiographic abnormalities;
RF, individuals with cardiovascular risk factors and normal left ventricular ejection fraction (LVEF);
ALVD, asymptomatic left ventricular dysfunction individuals with cardiovascular risk factors and abnormal left ventricular ejection fraction;
CHF, patients with chronic stable systolic heart failure.

All individuals were extensively phenotyped to check for clinical and biochemical parameters. Family history of coronary artery disease is defined as a family history of coronary event before 55 years in men and/or 65 years in women occurring in first degree relatives. * indicates P<0.05 for statistical comparison between RF and ALVD groups. = indicates P<0.05 for statistical comparison between ALVD and CHF groups. BNP, B-type natriuretic peptide.

### BNP Measurements

Healthy individuals had plasma BNP levels within the range 11±9 pg/ml. Plasma BNP levels were not statistically different between ALVD (27±23 pg/ml) and RF (15±10 pg/ml) patients, but were significantly increased in CHF (164±151 pg/ml) (see Table 1).

### Microarray Analysis

Blood samples were collected in 8 ml BD CPT vacutainer tubes that were processed immediately after collection according to the manufacturer's protocol. Total RNA were purified from collected white blood cell using RNeasy kit (Qiagen) in a Qiacube (Qiagen) automated protocol. Total RNA integrity was checked by capillary electrophoresis (Experion, Bio-Rad). Samples with RNA Quality Indicator≧8.5/10 were selected for analyses. Total RNAs were precisely quantified using RiboGreen and a Victor™ X5 2030 multilabel reader (Perkin Elmer). Total RNA (300 ng) were used for fluorescent labelling (QuickAmp Labelling, Agilent). Fluorescent RNAs were further purified on RNeasy columns. Since 3 groups were compared, we used the pooled reference sample protocol as a common reference to compare each sample common reference that was shown to obtain robust information (31). The labeled RNA were hybridized to pangenomic human glass microarrays from the consortium Reseau National des Genopole France and Medical Research Council, England; displaying 25,000 51-mer oligonucleotides probes. After standard hybridization, glass arrays were washed on a Ventana robotized apparatus and scanned using a GenPix 4000 scanner (Axon). Scanned images were processed by X-dot reader software with operator's validation of the spots detection.

### Statistical Analysis

Data normalization was performed in <<R>> (http://www.r-project.org/index.html) using R and the bioconductor package limma. Two-color Genepix files were normalized by using the “loess” method for within-array analyses and “Rquantile” for between-array comparisons, respectively. Five groups of 9 samples were established and used to calculate p-values with respect to the analysis of variance (aov) within each group. Log-ratios were extracted for probes in which the aov p-value was less than or equal to 0.001, resulting in 1055 probes before unsupervised statistical analysis by principal component analysis (PCA) with SIMCA P+ software (Umetrics). Class prediction was done by nearest centroids method using ClaNC software which is known to make the classifier more accurate by reducing the effect of noisy genes.

### Human Heart Samples

After ethical committee approval, all patients included in the sub-study gave their informed consent for sample collection and molecular analysis prior to their inclusion. Patients were carefully selected by the physicians from Department of Cardiology, Toulouse University hospital prior to cardiac surgery for coronary by-pass due to coronary disease. Samples from right auricle appendages were collected from the department of cardiovascular surgery of Toulouse University Hospital at the beginning of cardiac surgery and were of extra corporeal circulation. Samples were immediately washed in cold buffer, snap frozen in liquid nitrogen and maintained at −80° C. until analysis. Total RNA was isolated from the myocardium by using TRIzol reagent (Invitrogen, France) as described by the manufacturer. RNA integrity was checked by capillary electrophoresis using an Experion (Biorad) apparatus.

### Results

### ALVD Predictive Model Based on White Blood Cell Transcriptome

The inventors used the RNG-MRC 25k human pangenomic glass microarrays from the National Genopole Network to analyze blood gene expression of 25,341 genes. An unsupervised PCA analysis of the expression data was able to cluster patients into their respective groups: HI, RF, ALVD and CHF and revealed that blood gene expression profiles provide a molecular signature characteristic of ALVD. In order to build a ALVD predictive model, the inventors used the nearest centroid classification method (NCCM) from the ClaNC software package.

NCCM provided a set of genes whose expression profile led to a 100% successful classification of ALVD patients out of the 4 groups of individuals. They further tested the strength of our model of gene expression-based group prediction by leave-one-out cross-validation method. The classifier model accuracy and precision computed from the confusion matrix (Table 2) were 88% and 78%, respectively. The Fisher's exact test P-value (n=18, P=0.015) pointed out the robustness of the model.

TABLE 2
Leave-one-out's confusion matrix.

| | ALVD Individuals | RF Individuals |
|---|---|---|
| Classified as LVEF <45 | (true positive) | (false positive) |
| | 78% | 11% |
| Classified as LVEF ≧45% | (false negative) | (true negative) |
| | 22% | 89% |

Table 2 shows that classification performance of the nearest centroid classifier was determined using leave-one-out cross-validation method. Calculation of the classifier model accuracy and precision by standard formulae provided 83% and 87%, respectively. Fisher's exact test P-value (n=18) P=0.015.

### Additional ALVD Patient Group Validation

In addition to the original patient cohort, the inventors obtained blood samples from 8 additional ALVD and 8 RF individuals. These additional subjects fulfilled the ALVD parameter definition as they were in NYHA I class with an EF<45%, lacked HF symptoms, had low plasma BNP levels (25±11 pg/ml) which was not significantly different from the initial ALVD group (P=0.87) and were not matched for cardiovascular risk factors (Table 3). White blood cell gene expression analysis further validated using the ClaNC ALVD predictive model which gives 75% of true positive rate and 100% of true negative rate. Thus, the accuracy and precision of the prediction were 87% and 100%, respectively (Fisher's exact test P-value=0.007; n=16).

TABLE 3
Cardiovascular risk factors, clinical and biochemical
parameters of the ALVD validation group (n =
8) and RF individuals (n = 8).

| Groups | ALVD Individuals | RF |
|---|---|---|
| LVEF % | 39 (20-45) | 74 (65-81)* |
| Age (years) | 63 (75-40) | 67 (83-55) |
| Male % | 87 | 100 |
| Hypertensive % | 62 | 37 |
| Diabetes % | 12 | 25 |
| Obesity % | 0 | 12 |
| Dyslipidemia % | 50 | 37 |
| Heredity % | — | 0 |
| BMI | 25 ± 3 | 28 ± 5 |
| Systolic blood | 144 ± 19 | 145 ± 12 |
| pressure (mm Hg) | | |
| Diastolic blood | 82 ± 10 | 86 ± 8 |
| pressure (mm Hg) | | |
| Labs (mean ± s.d.) | | |
| BNP (pg/ml) | 25 ± 11 | 14 ± 9 |
| Na+ (mM) | 140 ± 2 | 140 ± 1 |
| creatinine (μM) | 110 ± 38 | 92 ± 12 |
| Hb (g/dl) | 14.0 ± 0.9 | 14.4 ± 0.4 |
| Leukocytes (cells/μl) | 6043 ± 1041 | 6682 ± 1360 |
| Lymphocytes (cells/μl) | 1433 ± 332 | 1959 ± 700 |
| Neutrophils (cells/μl) | 3975 ± 744 | 3049 ± 668 |

*indicates P < 0.05 for statistical comparison between RF and ALVD validation groups.

### Discriminant Genes

ClaNC defined a set of discriminant genes for the ALVD group including ALK, SLC43A2, NGFB, FBXW7, TMEM79, UBN1 and FECH (Table 4). Ingenuity's Pathway Analysis revealed that three genes encoded membrane proteins: the kinase ALK, TMEM79 and SLC43A2.

TABLE 4
ClaNC defined set of 7 discriminant genes for ALVD. Gene symbol,
Ensembl accession number, full gene name and protein location, as
provided by Ingenuity's Pathway Assist software, are indicated.

| Gene symbol | Ensembl accession number | Full gene name | Protein location |
|---|---|---|---|
| NGFB | ENSG00000134259 | Nerve growth factor (beta | Extracellular Space |
| | | polypeptide) | |
| TMEM79 | ENSG00000163472 | Transmembrane protein 79 | plasma membrane |
| FBXW7 | ENSG00000109670 | F-box and WD repeat | Nucleus; cytoplasm |
| | | domain containing 7 | |
| FECH | ENSG00000066926 | Ferrochelatase | Mitochondrion |
| ALK | ENSG00000171094 | Anaplastic lymphoma | plasma membrane |
| | | receptor tyrosine kinase | |
| UBN1 | ENSG00000118900 | Ubinuclein 1 | Nucleus |
| SLC43A2 | ENSG00000167703 | Solute carrier family 43, | plasma membrane |
| | | member 2 | |

FECH is a mitochondrial protein and FBXW7 encodes a cytoplasmic component of E3 ubiquitin protein ligase, which regulates the proteolytic machinery.

UBN1 is a transcription factor involved in the formation of senescence-associated heterochromatin foci.

NGFB (Nerve Growth Factor Beta) is a pleiotropic neurotrophin discovered over 50 years ago and involved in the development, maintenance of function and regeneration of nerve cells in the heart.

The inventors next looked for the pertinence of each of these genes with respect to ALVD subject classification. Box-and-whisker plots, which depict variations in gene expression levels, showed that:
expression of NGFB, TMEM79 and FBXW7 was significantly down-regulated in ALVD group (FIG. 2 a, b, c), whereas
expression of FECH, ALK, UBN1 and SLC43A2 were significantly increased in ALVD cases (FIG. 2 d, e, f, g).

A series of ROC curves were created for each of the proposed discriminant genes as related to the incidence of ALVD (FIG. 3).

Six genes out of seven (NGFB, TMEM79, FECH, FBXW7, ALK, UBN1) statistically differed from the null hypothesis (p<0.005 or p<0.0001, Table 5) and provided a good discrimination (no overlap in the two distributions) for both ALVD and CHF. SLC43A2 was an ALVD predictor but not a CHF predictor. The area under the curve (AUC), ranged from 0.59 to 0.92 for predicting ALVD and CHF. (Table 5).

TABLE 5
ROC curve statistical data. Area under curve (AUC), 95%
confidence interval (CI) and P values are indicated.

| Gene | AUC | 95% CI | P value |
|---|---|---|---|
| NGFB | 0.91 | 0.82-1.00 | <0.0001 |
| TMEM79 | 0.88 | 0.76-1.00 | <0.0001 |
| FBXW7 | 0.91 | 0.81-1.01 | <0.0001 |
| FECH | 0.92 | 0.82-1.02 | <0.0001 |
| ALK | 0.88 | 0.76-1.00 | <0.0001 |
| UBN1 | 0.78 | 0.62-0.94 | =0.0036 |
| SLC43A2 | 0.59 | 0.38-0.80 | =0.3589 |

### Expression in Human Heart

The inventors have further showed that UBN1, NGF and FECH genes displayed similar statistically significant regulation (up or down regulated) in hearts samples from heart failure patients but expression of the three remaining genes (TMEM79, FBXW7 and SLC43A2) could not be quantified, probably because of their low expression in heart. This is in accordance with other studies showing that white blood cells express a large number of heart genes but not all and that some genes expressions regulations in heart are also observed in white blood cells which might serve as surrogate markers for heart failure.

1-14. (canceled)

15. A method for diagnosing asymptomatic left ventricular systolic dysfunction in a subject comprising the steps of:
measuring, in a biological sample of the subject, a level of expression of at least one gene selected from the group consisting of NGFB, TMEM79, FBXW7, FECH, ALK, UBN1 and SLC43A2,
comparing the level of expression of the at least one gene with a corresponding reference level of expression of the at least one gene in at least one control sample; and
determining that said subject has asymptomatic left ventricular systolic dysfunction if the level of expression of the at least one gene differs from the corresponding reference level of expression.

16. The method of claim 15, wherein the step of measuring includes a step of
i) detecting a translation product of the at least one gene by
a) reacting the translation product with an antibody and detecting the formation of a complex between the translation product and the antibody;
or
b) using fluorescence-activated cell sorting;
and/or
ii) detecting a transcription product by extracting mRNA from the biological sample and hybridizing, reverse transcribing and/or amplifying extracted mRNA.

17. The method of claim 15, wherein the at least one gene includes a combination of at least 2, 3, 4, or 5 genes as listed in Table I.

18. The method of claim 15, wherein levels of expression of NGFB, TMEM79 and FBXW7 are down-regulated compared to corresponding reference levels of expression and levels of expression of FECH, ALK, UBN1 and SLC43A2 are increased compared to corresponding reference levels of expression

19. The method of claim 15, wherein the at least one control is a positive control and/or a negative control.

20. The method of claim 15, wherein the step of measuring measures translation products of the at least one gene.

21. The method of claim 20, wherein the translation products include proteins and/or polypeptides.

22. The method according claim 20, wherein the step of measuring is performed using fluorescence-activated cell sorting.

23. The method of claim 22, wherein the step of measuring measures transcription products of the at least one gene.

24. The method of claim 23, wherein the transcription products include mRNA.

25. The method of claim 15, wherein said step of measuring is performed using a DNA microarray.

26. The method of claim 25, wherein the DNA microarray includes at least one probe selected from the group consisting of SEQ ID NO: 1, SEQ ID NO: 2, SEQ ID NO: 3, SEQ NO: 4, SEQ ID NO: 5, SEQ ID NO: 6 and SEQ ID NO: 7.

27. The method of claim 15, further comprising a step of creating an expression profile of the at least one gene using at least one measured expression level of the gene.

28. The method according to claim 27, wherein the positive control is an expression profile of a subject suffering from chronic heart failure and/or an expression profile of a subject suffering from asymptomatic left ventricular systolic dysfunction.

29. The method according to claim 27, wherein the negative control is an expression profile of a healthy subject.

30. The method of claim 15, wherein the method further comprises a step of obtaining a transthoracic echocardiogram of the subject.


SEQUENCE LISTING (7 sequences)

SEQ ID NO: 1 - DNA, 50 bp, Artificial
  probe
gcactggaactcatattgtaccacgactcacacctttgtcaaggcgctga

SEQ ID NO: 2 - DNA, 50 bp, Artificial
  probe
ggctggggagtgcctggatggtgcggtggttgatgttaacctagtgtgtg

SEQ ID NO: 3 - DNA, 50 bp, Artificial
  probe
agctggtgtgtgcagttgggagtcggaatgggactgaagaaaccaagctg

SEQ ID NO: 4 - DNA, 51 bp, Artificial
  probe
aagcctctccatccagcatcttctctatcttagttgaatgccttctttctg

SEQ ID NO: 5 - DNA, 50 bp, Artificial
  probe
atgtttccttgccttgttgatgtggacatgagccatttgaggggagaggg

SEQ ID NO: 6 - DNA, 51 bp, Artificial
  probe
gcgactatgttggtgttaggggtggtgtggagattgttaatcttgtataaa

SEQ ID NO: 7 - DNA, 50 bp, Artificial
  probe
acacgttttcactgtcaccctcgttctgtctgccacctgaagggaatttg